(19) United States
(12) Patent Application Publication
(10) Pub. No.: US 2020/0315932 A1
(43) Pub. Date: Oct. 8, 2020

(54) STABLE, REVERSIBLE, COLORED PHOTOCHROMIC ADDITIVES AND METHODS OF MAKING THE SAME

(71) Applicant: KOEL COLOURS PVT. LTD., Mumbai (IN)

(72) Inventor: Dhirubhai Desai, Mumbai (IN)

(21) Appl. No.: 16/746,923

(22) Filed: Jan. 19, 2020

(30) Foreign Application Priority Data
Apr. 6, 2019 (IN) 201921013941

(51) Int. Cl. A61K 8/19; A61K 8/29; A61K 8/26; A61Q 17/04; C09C 1/00

(52) U.S. Cl. CPC A61K 8/19; A61K 8/29; A61K 8/26; C01P 2004/62; C09C 1/0084; A61K 2800/45; C01P 2006/60; A61Q 17/04

(57) ABSTRACT

A stable and consistent reversible coloured photochromic additive changes colour from original buff to desired colour shade between pink to blue when exposed to sunlight and restores to original colour in the absence of the sunlight; simultaneously having synergistic SPF value of at least 3. The additive comprises titanium dioxide, psuedoboehmite, iron oxide hydroxide and tungsten oxide.

### RELATED APPLICATIONS

This application claims priority under 35 U.S.C. § 119 to IN Application 201921013941, filed Apr. 6, 2019. This IN application is incorporated by reference herein in its entirety.

### BACKGROUND

### Field

The present invention relates in general to photochromic additives, particularly to a stable reversible coloured photochromic additive. The stable reversible coloured photochromic additive having SPF of at least 3 is an added advantage.

The present invention also relates to a process for the preparation of the stable reversible coloured photochromic additive, which is consistent in performance.

The present invention also relates to the use of the stable reversible coloured photochromic additive in various fields including cosmetics, personal care, healthcare, paint, ink, textile, glass, optical lenses, plastic, articles and the likes.

Stable and safe cosmetics with varying colourations are being continually explored and has become an important aspect to expand worldwide cosmetics business.

Cosmetic is an amazing tool to change or enhance ones appearance for the various purposes including self-expression, identification, ritual, special events and advertising. These cosmetics may include skin products including make-up and healthcare, hair product, paint and other types of products designed to alter the look of a person's face, amongst others. Recently, the evolution of colour changing make-up has driven Cosmetics industry to develop new cosmetic product with the photochromic pigments.

The property of changing colour when one substance is irradiated with light and then returning to its original colour, when such irradiation is stopped, is referred to as photochromic. For example, this is used in many applications including cosmetics, healthcare, personal care, glass, textile, ink, paint, toys, plastic, automotive, optical lenses, etc.

In the field of cosmetics, colour changing make-up which changes colour using photochromic material is known, particularly, it changes from original colour when indoors to changed colour when outdoors. Thus, photochromics change colour in response to sun light or black light. Photochromics are designed on the molecular level to interact with light or temperature or pH or moisture to maintain and evolve a desired colour shade. There are various materials either organic or inorganic, used to impart a particular desired cosmetic look in a particular condition. For any effective photochromic material, the molecular structure must contain delocalized electrons in the outermost shells which enable strong absorption in the visible part of the spectrum and imparting into predictable cosmetic colour shade.

The photochromic material may be organic and inorganic. Many organic photochromic materials have delocalized electrons in the outermost shell namely polycyclic aromatic hydrocarbons, coal tar products, azo dyes, quinolines, and the likes. However, some of the organic photochromic materials are often carcinogenic and thus may be undesirable or unacceptable for use in the cosmetics or healthcare. These organic photochromic materials penetrate the skin barrier and thus may increase a risk of toxicity and threat of health hazard. Most importantly, the organic pigments deteriorate in the sunlight as the time passes. Therefore, the organic pigment is not a preferred option.

The inorganic photochromic materials including metal oxides are well known. The advantage of the inorganic photochromic materials is that they are insoluble in water and lipid. The main advantage of the inorganic photochromic material, specifically non-nano material is that it does not penetrate the skin barrier. Thus, non-nano inorganic photochromic material is safe. In contrast to organic pigment, inorganic pigments does not deteriorate in the sunlight. Among inorganic metal oxides, titanium dioxide and iron oxide are two most widely used photochromic materials in the cosmetics industry.

Other class of metal oxides is transition metal oxide including tungsten oxide which is a well known photochromic material. Tungsten oxide (WO₃) is bright yellow in colour. When it is exposed to sun light, it changes its surface layer colour from bright yellow to dark blue. The colour change is due to transition from WO₃ to W₂O₅ in presence of blue-green light and IR light. But the reverse colour change to original colour is thermally driven or photo/light driven. It is used either alone or in combination with other metal oxides including but not limited to titanium dioxide, aluminium oxide and silicone dioxide as photochromic cosmetics.

Titanium oxide or a mixture of titanium oxide and iron oxide coated mica pigment has photochromic property. The make-up consisting the same illustrates darkening of the colour upon irradiation of light where intensity of light is strong, while it becomes original colour in the room where intensity of light is weak.

The cosmetic industry will keep on exploring for colour changing, particularly reversible colour changing cosmetic with different shades. At the same time the cosmetic should be stable and consistent in the performance. The applicant is exploring to provide the stable reversible coloured photochromic additive, when used in the cosmetic, changes the colour to a desired colour shade from pink to blue when irradiated with outdoor sunlight and then returning to its original colour when indoor. It is a challenge to provide the stable reversible coloured photochromic additive having enhanced strength of changed colour with stable and consistent performance when used in the cosmetic in the sunlight with SPF value of at least 3.

### SUMMARY

An object of the invention is to provide a stable reversible coloured photochromic additive comprising of titanium dioxide, psuedoboehmite, iron oxide hydroxide, tungsten oxide in a specific proportion; which changes colour from original buff colour to desired colour shade between pink to blue when exposed to sunlight and restores to original colour in the absence of the sunlight and simultaneously having synergistic SPF value of at least 3.

Another object of the invention is to provide the stable reversible coloured photochromic additive, which is safe and non-toxic as the particle size is of at least 200 nm which is non-nano.

Still another object of the invention is to provide a process for manufacturing the stable reversible coloured photochromic additive of the present invention. Yet another object of the invention is to provide a formulation comprising the stable reversible coloured photochromic additive of the present invention, which is suitable for cosmetics, personal care, healthcare, paint, ink, textile, plastic, glass, optical lense, article, automotive, toys and the likes.

Yet another object of the invention is to provide a cosmetic or personal care or healthcare composition comprising the reversible coloured photochromic additive of the present invention, which is safe, non-toxic, stable and consistent in performance and has an effective SPF value.

In the presently claimed invention, it is surprisingly found that the additive comprising titanium dioxide; psuedoboehmite; iron oxide hydroxide; and tungsten oxide in the ratio of 89.02 to 99.97:0.01 to 1:4.99 to 0.01:4.99 to 0.01 demonstrated colour change when exposed to sunlight and restored to its original colour when indoors with enhanced intensity and stable performance. The proportion of four components is very critical with respect to performance including enhanced intensity or strength of colour, stability and consistent performance. Stability and consistent performance is very important for any cosmetic formulation. At the same time, the additive is non-toxic being inorganic in nature as well as non-nano particle size leads to formation of carbon black pellets which simultaneously eliminates dusting and are easily disersible in aqueous medium leading to uniform dispersion of the carbon black pigment, enhancing Jetness and having blue tone which is ideal for end applications like cosmetics, paint, Ink and the likes.

Accordingly, in one embodiment, the presently claimed invention is directed to a stable and consistent reversible coloured photochromic additive; the additive comprises titanium dioxide; psuedoboehmite; iron oxide hydroxide; and tungsten oxide in the ratio of 89.02 to 99.97:0.01 to 1:4.99 to 0.01:4.99 to 0.01; the additive changes colour from original buff to desired colour shade between pink to blue when exposed to the sunlight and restores to original colour in the absence of the sunlight.

Typically, the additive comprises titanium dioxide; psuedoboehmite; iron oxide hydroxide; and tungsten oxide in the ratio of 94.5 to 94.9:0.1 to 0.5:4.99 to 0.01:4.99 to 0.01.

Typically, the mean particle size of the additive is in the range of 200 to 500 nm.

Typically the additive has synergistic SPF value of at least 3.

Accordingly, in another embodiment, the presently claimed invention is directed to a manufacturing process for the reversible coloured photochromic additive of the present invention;

the process comprises the following steps:
a. Mixing titanium dioxide, psuedoboehmite, iron oxide hydroxide and tungsten in a desired proportion followed by jet milling to obtain a uniform mixture with a mean particle size of 200 to 500 nm;
b. Calcinating the mixture obtained in step (a) in a muffle furnace at a temperature in the range of 800 to 1000° C. for at least 4 hours; and
c. Grinding the calcined product of step (b) by jet milling to get the reversible coloured photochromic additive with a mean particle size of at least above 200 nm and a synergistic SPF value of at least 3.

Typically, the proportion of titanium dioxide, psuedoboehmite, iron oxide hydroxide and tungsten oxide is in the ratio of 89.02 to 99.97:0.01 to 1:4.99 to 0.01:4.99 to 0.01.

Typically, the mixture of step (b) was calcinated in a muffle furnace at a temperature in the range of 800 to 900° C.

Typically, the jet milling of step (a) and (c) is carried out at a pressure of 1 to 8 bar and at a temperature 5 to 15° C.

Typically, the jet milling of step (a) and (c) is carried out at a pressure of 8 bar and at a temperature 8° C.

Accordingly, in yet another embodiment, the presently claimed invention is directed to a formulation comprising the reversible coloured photochromic additive of the invention and manufactured according to the invention; the formulation changes the colour in the presence of sun light and restore to the original colour in the absence of the sun light, including cosmetics, healthcare, personal care, ink, paint, textile, plastic, glass, optical lense, toys, automotive or article.

Accordingly, in yet another embodiment, the presently claimed invention is directed to a cosmetic or personal care or healthcare for skin, hair or nail care comprising the reversible coloured photochromic additive of the invention and manufactured according to the invention; the cosmetic or personal care or healthcare for skin, hair or nail care changes the colour in the presence of sun light and restore to the original colour in the absence of the sun light with SPF value of at least 3.

Typically, the cosmetic or personal care or healthcare for skin, hair or nail care is in the form of a lotion, cream, gel, emulsion or powder.

Accordingly, in yet another embodiment, the presently claimed invention is directed to an article comprising the reversible coloured photochromic additive of the invention and manufactured according to the invention; the article changes the colour in the presence of sun light and restore to the original colour in the absence of the sun light with SPF value of at least 3.

### DETAILED DESCRIPTION

The terms “a,” “an,” “the” and similar referents used in the context of describing the invention following claims are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Embodiments of the invention so claimed are inherently or expressly described and enabled herein.

As use herein, the term “photochromic” refers to a material, particularly inorganic material which changes original colour to different colour shade or darkens when exposed to sunlight.

As use herein, the term “reversible photochromic” refers to a material, particularly inorganic material which changes original colour to different colour shade or darkens when outdoors and return to original colour when indoors.

As used herein, the term “nano” refers to a median primary particle size that is smaller than 100 nano meters (i.e. nm).

As used herein, the term “non-nano” refers to a median primary particle size that is bigger than 100 nm. Particle size is calculated by measuring the particles using optical microscopy.

As used herein, the term “weight percent (wt. %)” when used without qualification, typically refers to the weight percent of a particular solid component as compared with all solid components present, excluding medium or vehicle.

In the following description, the embodiments are described in sufficient details to enable those skilled in the art to practice the invention. Other embodiments may be utilized and structural, logical and other changes may be made without departing from the spirit and scope of the present invention. The following detailed description is, therefore, not to be taken in a limiting sense. The detailed description that follows begins with a definition section followed by a description of various embodiments of the invention. A series of examples are presented later followed by a brief conclusion.

Most modern cosmetics contain titanium dioxide (TiO2) as well as iron oxide particularly, nano particles of TiO2 and iron oxide. These inorganic pigments blend more easily into the skin than that of their agglomerated form used earlier. These are known photochromic pigments as well as SPF additives. The cosmetic comprising the same shows change in the darkness when exposed to sunlight. There is also growing demand from the consumer or fashion industry for colour changing cosmetic with new shades, in particular enhanced shades, when exposed to sunlight and have original shades when it is used indoors simultaneously it should be safe and consistent in performance. The photochromic additive having acceptable SPF value of at least 3 will be an added advantage to the cosmetics industry. The reversible coloured photochromic additive has mean particle size of around 400 nm which makes the additive safe. We have carried out the search and found that there is no additive present in the existing art which discloses the reversible coloured photochromic additive of the present invention.

According to the present invention, there is provided a stable reversible coloured photochromic additive which changes original buff colour to desired colour shade between pink to blue when exposed to the sunlight and restores to the original colour in the absence of sunlight i.e. indoor and simultaneously having synergistic SPF value of at least 3. It typically comprises titanium dioxide; psuedoboehmite; iron oxide hydroxide; and tungsten oxide in the ratio of 89.02 to 99.97:0.01 to 1:4.99 to 0.01:4.99 to 0.01. Preferably, the additive comprises titanium dioxide; psuedoboehmite; iron oxide hydroxide; and tungsten oxide in the ratio of 94.5 to 94.9:0.1 to 0.5:4.99 to 0.01:4.99 to 0.01.

The additive of the present invention has a mean particle size of at least above 200 nm. In particular, the mean particle size of the additive is in the range of 200 to 500 nm; more particularly around 300 to 400 nm.

The additive of the present invention comprising more of iron oxide hydroxide changes original colour to desired colour shade between pink to red when exposed to sunlight and restores to original colour in the absence of the sunlight and simultaneously having synergistic SPF value of at least 3.

The additive of the present invention comprising more of tungsten oxide changes the original colour to desired colour shade between violet to blue when exposed to sunlight and restores to the original colour in the absence of the sunlight and simultaneously having synergistic SPF value of at least 3.

Similarly, the proportion of iron oxide hydroxide and tungsten oxide may be varied to achieve the desired colour shade between pink to blue in sunlight. Psuedoboehmite, iron oxide hydroxide and tungesten oxide are required at particular concentration to achieve stable reversible coloured photochromic additive of the present invention to change the original buff colour with desired colour shades with high intensity/strength in the presence of sunlight and restore to the original colour in absence of the sunlight. This also gives consistency in the performance to the additive of the present invention.

According to the present invention, there is provided a manufacturing process for the reversible coloured photochromic additive of the present invention. This process comprises the following steps:
a. Titanium dioxide, psuedoboehmite, iron oxide hydroxide and tungsten oxide were admixed in a desired proportion and it was milled further by jet milling to obtain uniform mixture having a mean particle size of 200 to 500 nm;
b. The mixture was calcinated in a muffle furnace at a temperature in the range of 800 to 1000° C. for at least 4 hours; and
c. The calcined product was ground by jet milling to get the reversible coloured photochromic additive having a mean particle size of at least above 200 nm and having a synergistic SPF value of at least 3.

Typically, the proportion of titanium dioxide, psuedoboehmite, iron oxide hydroxide and tungsten oxide of step (a) may be varied based on the desired colour shades required in the sunlight.

Typically, the proportion of titanium dioxide, psuedoboehmite, iron oxide hydroxide and tungsten oxide is in the ratio of 89.02 to 99.97:0.01 to 1:4.99 to 0.01:4.99 to 0.01. Preferably, the proportion is 94.5 to 94.9:0.1 to 0.5:4.99 to 0.01:4.99 to 0.01.

Typically, the mixture of step (b) was calcinated in a muffle furnace at a temperature in the range of 800 to 900° C., more preferably at 840° C.

Typically, the jet milling of step (a) and (c) is carried out at a pressure of 1 to 8 bar and at a temperature 5 to 15° C., particularly, the jet milling of step (a) and (c) is carried out at a pressure of 8 bar and at a temperature 8° C.

Typically, the mean particle size of the additive so obtained in the step (c) is of at least 200 nm; particularly in the range of 200 to 500 nm; more particularly at around 400 nm.

According to the present invention, there is provided a formulation comprising the reversible coloured photochromic additive of the present invention. This formulation is suitable for many applications, which requires change in the colour in the presence of sun light and restore to the original colour in the absence of the sun light, including cosmetics, healthcare, personal care, ink, paint, textile, plastic, glass, optical lense, toys, automotive, article and the likes.

According to the present invention, there is provided a cosmetic or personal care or healthcare for skin, hair or nail care comprising the reversible coloured photochromic additive of the present invention and specifically used for skin, hair or nail care. Besides colour change, the cosmetic or personal care or healthcare has SPF value of at least 3. Preferably, the cosmetic or personal care or healthcare is in the form of a lotion, cream, gel, emulsion, powder depending on the bases used therein. Preferably, it is in the form of cream, lotion or powder. It is used in the sunscreen, sun-lotion, foundation, lip-stick, lip-glows, lip balms, nail polish, hair colour, eyeliner, eye shadow and pressed powder.

Typically, the cosmetic or personal care or health care of the present invention, further comprising UV filter agent or a topically active agent selected from an anti tanning agent, an antimicrobial agent, a depigmentation agent, an anti aging agent, antifungal agent, an insect repellent and a combination thereof.

The cosmetic or personal care or healthcare compositions of the present invention may further comprise additional additives like antioxidants, binders, biological additives, buffering agents, colourants, thickeners, polymers, astringents, fragrance, humectants, opacifying agents, conditioners, exfoliating agents, pH adjusters, preservatives, natural extracts, essential oils, skin sensates, skin soothing agents, skin healing agents, or the like.

An article comprising the reversible coloured photochromic additive of the present invention.

Thus, the present invention facilitates the stable reversible coloured photochromic additive which comprises titanium dioxide, psuedoboehmite, iron oxide hydroxide and tungsten oxide, where psuedoboehmite may act as booster and excites delocalised electron of titanium dioxide, iron oxide hydroxide and tungsten oxide in the presence of sun light from valence bond to covalent bond. This causes shift in the colour from original buff colour to any shade between pink to red and blue. When the sunlight exposure is removed, the excited electron again come back to the valence bond. Thus, the presence of psuedoboehmite in the additive enhances strength of the changed colour shades and the photochromic behaviour of the additive upon exposure to sunlight. The median primary particle size of the additive of the present invention is of at least 200 nm that makes the additive safe and non toxic to human as well as aquatic eco-system. SPF value of the additive of the present invention is of at least 3, which is an added advantage of the present invention. The synergistic characteristic of the reversible coloured photochromic additive of the present invention is useful in the cosmetics or personal care or healthcare or the formulations of glass, paint, automobile, toys, ink, textile, plastic, optical lense or articles that require colour change at outdoors for the aesthetic cause or to improve look with or without additional benefits of SPF.

### Chemicals Used

1. Titanium dioxide (Anatase) (TiO₂: 97-98%; Tinting Strength: 1220 min; Specific Gravity 3.8-3.9; Refractive Index 2.5; pH: 6.5-8; Bulk Density (tamped): 0.7±0.2 gm/cm³, Moisture: 0.4% max.; Oil Absorption: 20-25 gm/100 gm; and Average Particle Size: 300 to 700 nm)
2. Aluminium oxide-hydroxide (CAS No: 1333-84-2: pore volume of 0.8 to 1.0 g/ml and minimum surface area of 250 m²/g, particle size of 300 to 400 nm)
3. Iron oxide hydroxide (particle size of 300 to 400 nm)
4. Tungsten oxide (particle size of 300 to 400 nm)

### EXAMPLE

We have exemplified the formulae of the stable reversible coloured photochromic additive of the invention in Table 1.

TABLE 1
The stable reversible coloured photochromic additive (i.e. RCPA) Formulae

| | RCPA | | | | | | | | |
|---|---|---|---|---|---|---|---|---|---|
| | RCPA | RCPA | RCPA | RCPA | RCPA | RCPA | RCPA | RCPA | RCPA |
| Components | (1) | (2) | (3) | (4) | (5) | (6) | (7) | (8) | (9) |
| Titanium | 94 | 95 | 94.5 | 94 | 95 | 94.5 | 94.9 | 97 | 97 |
| dioxide | | | | | | | | | |
| (Anatase) | | | | | | | | | |
| (A) % | | | | | | | | | |
| Aluminium | 1 | 0 | 0.5 | 1 | 0 | 0.5 | 0.1 | 0.1 | 0.1 |
| oxide- | | | | | | | | | |
| hydroxide | | | | | | | | | |
| (B) % | | | | | | | | | |
| Iron oxide | 4.99 | 4.99 | 4.99 | 0.01 | 0.01 | 0.01 | 4.99 | 2.9 | 0.01 |
| hydroxide | | | | | | | | | |
| (C) % | | | | | | | | | |
| Tungsten | 0.01 | 0.01 | 0.01 | 4.99 | 4.99 | 4.99 | 0.01 | 0.01 | 2.9 |
| oxide | | | | | | | | | |
| (D) % | | | | | | | | | |

Process:
1. The components A, B, C and D were mixed in various proportions as given in Table 1 by jet milling at 8 bar and maintaining temperature at 8° C.;
2. The mixtures were calcined at 840° C. for 4 hours;
3. The calcined products i.e. additives were cooled and further jet milled at 8 bar and maintaining temperature at 8° C. to obtain additives 1 to 9.

The additives (1) to (9) were first tested for their SPF value by COLIPA 2011 and mean particle size by ASTM E2651-13 (Particle size analysis). The results of SPF and particle size are illustrated in Table 2.

TABLE 2
Test Results of the Additives (1) to
(9) for SPF and mean particle size

| RCPA | SPF Value | Mean particle size |
|---|---|---|
| RCPA (1) | 3 | 400 nm |
| RCPA (2) | 3 | 400 nm |
| RCPA (3) | 3 | 400 nm |
| RCPA (4) | 3 | 400 nm |
| RCPA (5) | 3 | 400 nm |
| RCPA (6) | 3 | 400 nm |
| RCPA (7) | 3 | 400 nm |
| RCPA (8) | 3 | 400 nm |
| RCPA (9) | 3 | 400 nm |

CIELAB color space (1976) was used to measure colour and its strength. It expresses color as three values: L* for the lightness from black (0) to white (100), a* from green (−) to red (+), and b* from blue (−) to yellow (+). CIELAB was designed so that the same amount of numerical change in these values corresponds to roughly the same amount of visually perceived change. The colour of the additives without exposing sunlight was measured and used as standard for comparison purpose. The additives (1) to (9) were exposed to sunlights leading to colour change. The change in the colour and its intensity was measured for each additives and compared with standards. The values of L*, a* and b* are illustrated in Table 3.

TABLE 3
Results of CIELAB

| RCPA | (1) | (2) | (3) | (4) | (5) | (6) | (7) | (8) | (9) |
|---|---|---|---|---|---|---|---|---|---|
| a* | 0.426 | 0.116 | 0.777 | −0.112 | −0.233 | 0.439 | 0.6 | 0.16 | 0.036 |
| b* | −0.567 | −0.521 | 0.184 | −0.465 | −0.334 | −0.777 | −0.51 | 0.17 | −0.51 |
| L* | −0.176 | −0.63 | 0.05 | −0.116 | −0.034 | −0.286 | −0.07 | 0.04 | −0.17 |
| Intensity | 105.213 | 103.3 | 120.8 | 117.86 | 117.337 | 137.96 | 99.97 | 99.2 | 139.61 |

According to Table 3, the additive having Iron oxide in the range of 0.01 to 5% changes colour from its original buff colour to range of shades from pink to red at different concentrations of which was reflecting photochromic behaviour. At the same time, intensity of changed colour was maximum at 0.5% of psuedoboehmite and more reddish with 0.01% of tungsten oxide. Similarly, in case of the additive having tungsten oxide in the range of 0.01 to 5% changes colour from its original buff colour to blue shades at different concentrations of which was reflecting photochromic behaviour. At the same time, intensity of changed colour was maximum at 0.1 to 0.5% of psuedoboehmite and more bluer with 0.01% of iron oxide. These results prove the synergistic property of additive with intense change in the colour shade and thus better photochromic behaviour than the individual. The performance was found to be stable and consistent. The additive also showed SPF of 3 which was added advantage. All additives reflect change of colour in presence of sunlight and restore to its original colour when indoors.

Thus, the additive of the presently claimed invention was safe and non toxic to human as well as aquatic eco-system as it was non-nano. It was converted into the cosmetic composition. It was useful in the end applications including lotion, cream, foundation, lip-stick, nail polish and pressed powder.

The present invention has been described with reference to preferred embodiments, purely for the sake of understanding and not by way of any limitation and the present invention includes all legitimate developments within the scope of what has been described hereinbefore and claimed in the appended claims.

We claim:

1. A stable, reversible, colored photochromic additive comprising:
titanium dioxide in approximately 89.02-99.97 wt %;
psuedoboehmite in approximately 0.01-1 wt %;
iron oxide hydroxide in approximately 4.99-0.01 wt %; and
tungsten oxide in approximately 4.99-0.01 wt %, wherein the additive changes from a first color to a second color when exposed to sunlight, and wherein the additive reverts to the first color from the second color in the absence of the sunlight.

2. The additive of claim 1, wherein,
the titanium dioxide is approximately 94.5-94.9 wt %, and
the psuedoboehmite is approximately 0.1-0.5 wt %.

3. The additive of claim 1, wherein the additive is in a calcined form, and wherein the mean particle size of the additive is approximately 200-500 nm.

4. The additive of claim 1, wherein the additive has a SPF value of at least 3.

5. The additive of claim 1, wherein the first color is buff and the second color is between pink and blue on the color spectrum.

6. A method of manufacturing a stable, reversible, colored photochromic additive of titanium dioxide in approximately 89.02-99.97 wt %, psuedoboehmite in approximately 0.01-1 wt %, iron oxide hydroxide in approximately 4.99-0.01 wt %, and tungsten oxide in approximately 4.99-0.01 wt %, the additive changing from a first color to a second color when exposed to sunlight and reverting to the first color from the second color in the absence of the sunlight, the method comprising:
mixing titanium dioxide, psuedoboehmite, iron oxide hydroxide, and tungsten oxide into a mixture of approximately 89.02-99.97 wt % titanium dioxide, approximately 0.01-1 wt % psuediboehmite, approximately 4.99-0.01 wt % iron oxide hydroxide, and approximately 4.99-0.01 wt % tungsten oxide;
jet milling the mixture to obtain a uniform mixture with a mean particle size of approximately 200 to 500 nm;
calcinating the uniform mixture in a muffle furnace at a temperature in the range of approximately 800 to 1000° C. for at least 4 hours; and
jet milling the uniform mixture following the calcinating to produce the additive with a mean particle size of at least 200 nm.

7. The method of claim 6, wherein the additive is titanium dioxide in approximately 94.5-94.9 wt % and psuedoboehmite in approximately 0.1-0.5 wt %.

8. The method of claim 6, wherein the muffle furnace calcinates at a temperature of approximately 800 to 900° C.

9. The method of claim 6, wherein the jet millings are carried out at a pressure of approximately 1 to 8 bar and at a temperature of approximately 5 to 15° C.

10. The method of claim 8, wherein the jet millings are carried out at a pressure of approximately 8 bar and at a temperature of approximately 8° C.

11. The method of claim 6, wherein the mean particle size of the additive is approximately 200-500 nm.

12. The method of claim 6, wherein the first color is buff and the second color is between pink and blue on the color spectrum.

13. The method of claim 6, wherein the additive has a SPF value of at least 3.

14. A formulation comprising:
an additive including titanium dioxide in approximately 89.02-99.97 wt %, psuedoboehmite in approximately 0.01-1 wt %, iron oxide hydroxide in approximately 4.99-0.01 wt %, and tungsten oxide in approximately 4.99-0.01 wt %, wherein the additive changes from a first color to a second color when exposed to sunlight, and wherein the additive reverts to the first color from the second color in the absence of the sunlight; and
a cosmetic, therapeutic, ink, paint, textile, plastic, glass, optical lens, or automotive carrier.

15. The formulation of claim 14, wherein the carrier is a cosmetic or therapeutic carrier for application on skin, hair, or nails, and wherein the additive has a SPF value of at least 3.

16. The formulation of claim 15, wherein the carrier is at least one of a lotion, cream, gel, emulsion, and powder.

17. The formulation of claim 14, wherein the additive includes titanium dioxide in approximately 94.5-94.9 wt %, and psuedoboehmite in approximately 0.1-0.5 wt %.

18. The formulation of claim 14, wherein the mean particle size of the additive is approximately 200-500 nm.

19. The formulation of claim 14, wherein the first color is buff and the second color is between pink and blue on the color spectrum.

20. The formulation of claim 14, wherein the additive is in a calcined form.
